(19) United States
(12) Patent Application Publication
(10) Pub. No.: US 2006/0130162 A1
(43) Pub. Date: Jun. 15, 2006

(54) Shrimp and the production thereof

(76) Inventors: David J. Kyle, Catonsville, MD (US); Robert A. Bullis, Ellicott City, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/532,344

(22) PCT Filed: Oct. 24, 2003
(86) PCT No.: PCT/US03/31518

§ 371 Date: Sep. 2, 2005

Related U.S. Application Data
(60) Provisional application No. 60/420,789, filed on Oct. 24, 2002.

(51) Int. Cl. A01K 67/033

(52) U.S. Cl. 800/13

(57) ABSTRACT

This invention is directed to compositions comprising improved edible shrimp and other crustaceans. The shrimp or other crustaceans are enriched with one or more components that provide benefits, such as improved taste and/or appearance, or improved health benefits for humans consuming them. The invention also relates to methods of producing such shrimp and other crustaceans, and methods of making a feed to feed the shrimp and other crustaceans.

### PRIORITY CLAIM

This application claims the priority of provisional application 60/420,789, which was filed in the United States Patent and Trademark Office on Oct. 24, 2002, the disclosure of which is herein incorporated by reference.

### TECHNICAL FIELD

The invention relates to shrimp raised in aquaculture, and the methods employed to raise the shrimp. The shrimp of the invention provide an improved taste and appearance, and provide improved health benefits to the humans who consume them.

### BACKGROUND ART

Commercially available shrimp have nutritional attributes that arc not optimal for humans. Shrimp have a high level of cholesterol and a lower level of polyunsaturated fatty acids (e.g., docosahexaenoic acid or DHA) than are optimal for human nutrition in the commercial shrimp currently on the market (Middleditch et al. 1980). There is general concern that eating a food with very high levels of cholesterol will elevate the consumer's level of cholesterol. Most studies indicate that, in normal healthy individuals, eating seafood high in cholesterol is “mildly hyperlipidemic” (Connor and Lin 1982; Childs et al. 1990; De Oliveira e Silva et al. 1996). However, one study showed that eating shellfish may improve the lipid profile of humans (Tanaka et al. 1998), but this may be due to the presence of small amounts of omega-3 fatty acids consumed along with the extra cholesterol in the shellfish. In general, however, a lower cholesterol shrimp would be a preferred food source to lower dietary cholesterol intake.

One problem with crustaceans, especially shrimp, grown in aquaculture systems is subtle changes in their taste compared to marine-raised animals. Freshwater grown shrimp have an even greater problem with taste compared to sea caught animals and lack, the sufficient consumer appeal to compete effectively with the wild catch. Therefore, growth in acceptance and consumption of aquacultured species may be impeded by subtle flavors that are considered by many as less desirable than those of traditional wild-caught counterparts. Both consumers and experts have noted that aquacultured species, including shrimp, lack the rich, sea-like flavors that are found in wild-caught stocks (Sylvia and Graham 1991; Kummer 1992). Volatile bromophenols are widely distributed in marine fish, crustaceans, and mollusks, but are virtually absent in freshwater species (Boyle et al. 1992a; Boyle et al. 1992b). Recently, low concentrations of volatile bromophenols have been recognized as key components in providing desirable sea-like and iodine-like flavors to seafoods (Boyle et al. 1992b). To our knowledge, no one has attempted to utilize these compounds as flavor enhancers in defined crustacean diets (especially shrimp).

Because of consumer concerns about the appropriateness of wild-caught seafood for human consumption (due to environmental damage and over exploitation of natural resources), as well as the perceived inferiority of animals raised on diets containing ingredients derived from possibly pollution-contaminated (e.g., mercury, pesticides, pathogens, antibiotic residues, and industrial pollutants) and declining wild resources (e.g., fishmeal and fish oil), there is a growing preference for “Organic” foods which are certified to not contain substances of suspicious origin. Organic certification requires that the feeds provided to aquacultured species meet the requirements of the Organic Foods Protection Act (USDA 2002b), which states that the total feed ration be composed of products that are Organically produced, and if applicable, Organically handled. To our knowledge, there does not appear to be a shrimp diet on the market that can meet the requirements for Orgainic certification. Indeed, most of the shrimp presently marketed are either wild catch or produced in semi-intensive production systems that would not qualify for Organic certification. Particularly problematic is the necessity of using fishmeal and/or fish oil in the feed for optimal growth of the animals (especially shrimp and carnivorous fish).

Carotenoids (e.g., astaxanthin) are responsible for the desirable body coloration in shrimp. Appearance, both before and after cooking, is an important factor influencing the consumers' purchase of seafood products. Such carotenoids can be of direct dietary origin or derived from metabolic transformation of another dietary carotenoid (Meyers and Latscha 1997). The principal carotenoid in shrimp is astaxanthin and this compound is typically added to shrimp feeds. Wild animals typically obtain astaxanthin from microalgae, which are in the diet either directly or indirectly. Other xanthophylls (such as lutein or zeaxanthin) and other carotenes (such as lycopene or γ-carotene) are typically not found in shrimp to any great extent. In humans, lutein and zeaxanthin have recently been associated with better eye health, and the occurrence of age related macular degeneration (AMD) has been inversely correlated with dietary lutein and/or zeaxanthin. Thus, the replacement of the astaxanthin or the enrichment of the shrimp in lutein and zeaxanthin would provide a food product that carries an additional eye health benefit to the consumer.

Shrimp, like other marine animals, contain omega-3 long chain polyunsaturated fatty acids (LC-PUFAs). LC-PUFAs, such as DHA, have recently been shown to have a significant health benefit to growing infants, nursing mothers, children, and adults in general (Horrocks and Yeo 1999). Therefore, increased consumption of DHA is recommended to compensate for the ω-3 oil-deficiency in the modem diet (Uauy-Dagach and Valenzuela 1992). Terrestrial plant and animal based foods are relatively deficient in omega-3 LC-PUFAs and are enriched in omega-6 LC-PUFAs. The main source in modern human diets is certain fatty fish, such as salmon and tuna, which are rich sources of DHA for humans. However, in 2002, shrimp surpassed tuna as the most preferred seafood product in the United States. Shrimp, on the other hand, are not a rich source of DHA. Thus, it would be of great health benefit to provide a source of shrimp that would deliver DHA in levels equivalent to or exceeding that of other oily fish. Prior to this invention, such high-DHA shrimp or shrimp-products have never been obtained.

Custom designed crustacean feeds and production methods that enable the production of shrimp and other crustaceans (such as lobster, crayfish, and crab) that have a better taste than other aquaculture raised shrimp, improved LC-PUFA profiles that enhance consumer health (e.g., increase in DHA and/or decrease in cholesterol), and a distinct, pleasing visual profile are novel, thereby delivering a significant improvement over existing commercially available shrimp. As the wild catch decreases, such Designer Shrimp will fill an increasing market demand while improving on the nutritional value delivered over existing marine-raised shrimp.

As the wild-caught seafood species are exposed to increasing levels of overall pollution, the consumption of these as food becomes a public health issue. However, the governmental recommendations against eating certain fish species due to health risks due to pollutants are being countermanded by governmental recommendations to increase the intake of polyunsaturated fatty acids important to human health. This is especially important for pregnant women and infants, with correlations to good health and DHA content firmly established. This presents a public health conundrum that this invention will be able to solve by providing improved seafoods that have higher levels of healthy components (e.g., carotenoids, DHA, ARA) and lower or negligible levels of pollutants.

### DISCLOSURE OF THE INVENTION

This invention is directed to the production and use of edible shrimp or other cultured crustaceans (such as but not limited to crab and lobster) that are highly enriched with one or more compounds that are deemed to be of health benefit to humans consuming the shrimp or other crustaceans. In particular, a shrimp product can be enriched in docosahexaenoic acid (DHA), certain carotenoids, such as lutein, certain flavor enhancing compounds, such as 2,6-dibromophenol or 2,4,6-tribromophenol, and/or depleted in cholesterol. Methods are also contemplated to enable Organic certification of shrimp by replacement of undefined components with defined components, such as microalgal biomass.

### DEFINITIONS

In describing the present invention, the following terminology is used in accordance with the definitions set out below.

An “Organic Shrimp” is a shrimp raised in such a way that 95% of the components in the feeds utilized for production are from certified Organic sources. The production process used for these Organic shrimp controls the inputs and outputs of the production system to minimize the impact of shrimp production on the environment. Note: for the purposes of this patent application capitalization is used to differentiate the chemical use of organic from the statutory use of Organic, as defined herein.

A “100% Organic Shrimp” is a shrimp raised in such away that 100% of the feeds utilized for production are from certified 100% Organic sources. The production process used for these Organic shrimp controls the inputs and outputs of the production system to minimize the impact of shrimp production on the environment.

A “Finishing Feed” is a feed that is provided to an animal prior to harvest and not during the full course of production. This can be preferably as short as 1 day but can be up to two months in the case of crustaceans, especially shrimp.

A “Flavor Enhancer” is defined as any compound, inorganic or organic, that is added to a feed to improve the flavor of the final product. As it pertains to this invention, Flavor Enhancing compounds can include, but are not limited to, bromophenol, 2,6-dibromophenol, 2,4,6-tribromophenol, and iodine.

A “crustacean” and the plural “crustaceans” are defined as any member of the Class Crustacea, such as, but not limited to, shrimp, lobsters, red claws, and crabs.

A “shrimp” and the plural “shrimp” are defined as a crustacean or crustaceans generally referred to as shrimp, prawn, or langostina, such as, but not limited to, members from the following genera: Penaeus, Litopenaeus, Pandalus, Macrobrachium, Crangon, Cherax, and Metapenaeus.

“Microalgae” refers to prokaryotic and eukaryotic algae that are classed in many different species. Normally the prokaryotic algae are referred to as cyanobacteria or bluegreen algae. The eukaryotic microalgae come from many different genera, some of which overlap with the macroalgae and are differentiated from these by their size and a lack of defined organs (although they do have specialized cell types). Examples of different groups containing microalgae follow but are not limited to the chlorophyta, rhodophyta, phaeophyta, dinophyta, euglenophyta, cyanophyta, prochlorophyta, and cryptophyta.

A “carotenoid” encompasses any molecule in a class of yellow to red pigments, including carotenes and xanthophylls. “Carotenes” are orange-yellow to red pigments that are found in some animal tissues and plants, and may be converted to Vitamin A in the liver. “Xanthophylls” are yellow pigments, some of which may be found with chlorophyll in green plants.

An “aquaculturally-raised” shrimp is any shrimp that was cultivated, either in freshwater or saltwater, where the shrimp are contained in an artificially imposed system. Such system would allow one to apply aspects of animal husbandry specific to this group of crustaceans that allow controlled growth and harvesting of the shrimp for use, processing, and/or sale.

### EMBODIMENTS OF THE INVENTION

In one embodiment, the present invention provides a shrimp or other crustacean, which has been selectively enriched with certain health beneficial compounds, such as, but not limited to, LC-PUFAs (e.g., DHA, ARA or arachidonic acid, EPA), carotenoids (e.g., lutein, β-carotene, astaxanthin, zeaxanthin, γ-carotene), vitamins (e.g., vitamin A, vitamin C, vitamin E), minerals (e.g., iron, zinc, selenium, magnesium), or other beneficial compounds. These are referred to herein as “designer” shrimp or other crustaceans.

In another embodiment, the present invention provides a method to culture shrimp or other crustaceans selectively enriched with certain health beneficial compounds, such as, but not limited to, LC-PUFAs (e.g., DHA, ARA, EPA), carotenoids (e.g., lutein, β-carotene, astaxanthin, zeaxanthin), vitamins (e.g., vitamin A, vitamin C, vitamin E), minerals (e.g., iron, zinc, selenium, magnesium), or other beneficial compounds.

In the embodiments described above, the invention provides animals with improved nutritional qualities and reduced or negligible levels of pollutants or undesirable compounds commonly found in nature. In this way, the risk of consumption by the final consumer is significantly reduced (i.e., a much healthier shrimp).

The invention provides aquaculturally raised shrimp comprising a DHA level higher than about 12.5 μg/g, more preferably higher than about 25 μg/g, or most preferably higher than 50 μg/g fresh weight. The invention also provides an aquaculturally-raised shrimp comprising carotenoids, wherein astaxanthin comprises less than about 80% of the total carotenoids. The remaining non-astaxanthin carotenoids can comprise one or more of the following: β-carotene, γ-carotene, lutein, lycopene, zeaxanthin, and canthaxanthin. The invention provides aquaculturally raised shrimp with a lutein level higher than about 5 μg/g fresh weight.

The invention provides aquaculturally raised shrimp comprising one or more Flavor Enhancer (e.g., bromophenol, 2,6-dibromophenol, 2,4,6-tribromophenol, and/or iodine). The invention provides that the 2,6-dibromophenol levels can be higher than about 0.06 μg/ kg fresh weight. The invention provides that the 2,4,6-tribromophenol levels can be higher than about 6 μg per kilogram fresh weight. The invention also provides an aquaculturally raised shrimp comprising 2,6-dibromophenol at a level higher than about 0.06 μg/kg and 2,4,6-tribromophenol at a level higher than about 6 μg/kg fresh weight.

The invention provides aquaculturally raised shrimp comprising cholesterol at a level lower than about 8.0 mg/g, more preferably lower than about 6 mg/g, and most preferably lower than about 1 mg/g fresh weight. The invention also provides, aquaculturally raised shrimp comprising a DHA/EPA ratio greater than about 2.0, more preferably greater than about 2.5, or most preferably greater than about 5.0. The invention further provides an aquaculturally-raised shrimp fed an exclusively vegetarian diet comprising hydrolyzed plant protein and microalgae.

The invention provides an Organic shrimp. The invention also provides a 100% Organic shrimp. The invention provides an aquaculturally raised shrimp that has been certified as Organic by the United States Department of Agriculture (USDA). The invention most preferably provides an aquaculturally raised shrimp that has been certified as 100% Organic by the United States Department of Agriculture.

In a further aspect, the invention provides a shrimp feed comprising red rice yeast. The invention provides a whole Monascus sp. biomass, a lysed Monascus sp. biomass, and fractions of a whole Monascus sp. biomass and a lysed Monascus sp. biomass. The Monascus can be from a number of different species such as Monascus purpureus.

In an aspect, the invention provides a feed for shrimp. This feed can comprise a high level of DHA, lutein, lycopene, zeaxanthin, and/or bromophenols. The invention provides that the DHA level is greater than 12.5 μg per gram fresh weight. The invention also provides that the lutein level is greater than 5 μg per gram fresh weight. The invention also provides that the lycopene level is greater than 5 μg per gram fresh weight. The invention also provides that the zeaxanthin level is greater than 6 μg per gram fresh weight. The shrimp feed can also comprise a low level of chlorophyll.

The invention further provides a shrimp feed comprising 2,6-dibromophenol at a range from about 10 to about 1000 mg/kg. The invention also provides a shrimp feed comprising 2,4,6-tribromophenol at a range from about 10 to about 1000 mg/kg. The invention further provides a shrimp feed comprising 2,4,6-tribromophenol at a range from about 10 to about 1000 mg/kg and 2,6-dibromophenol from about 10 to about 1000 mg/kg.

In another aspect, the invention provides methods of producing shrimp. The invention provides a method of producing an Organic shrimp by feeding microalgal DHA to the shrimp. The invention also provides a method of producing a shrimp containing high levels of DHA by feeding the shrimp one or more microalgae and/or microalgal extracts enriched in DHA. The microalgae can be chosen from dinoflagelates including, but not limited to, Crypthecodinium sp., Crypthecodinium cohnii, chitrids including, but not limited to, Schizochytrium sp., Schizochytrium aggregatum, Schizochytrium aggregatum ATCC 28209, Thraustochytrium roseum ATCC 28210, Thraustochytrium sp. ATCC 26185, Thraustochytrium sp., Thraustochytrium visurgense ATCC 28208, Ulkenia sp., and microalgae, including but not limited to, Pavlova sp., Tetraselmis sp., and Isochrysis sp.

The invention provides a method of producing a shrimp with a high carotenoid level, comprising providing the shrimp with a feed comprising a biomass enriched in one or more carotenoid. The carotenoid can be chosen from, e.g., lutein, lycopene, and zeaxanthin. The sources of the carotenoid can be chosen from one or more of microalgae, marigold extract, marigold petals, tomato extract, and processed tomato biomass.

The invention provides a method of increasing the desirability of the flavor profile of a shrimp by adding one or more Flavor Enhancers to the shrimp's feed. These Flavor Enhancers can be bromophenols (e.g., 2,6-dibromophenol and/or 2,4,6-tribromophenol).

The invention also provides a method of feeding a shrimp to a human or non-human animal, comprising providing for the animal's consumption, a high-DHA shrimp, a high-carotenoid shrimp, a low cholesterol shrimp, and/or an Organic shrimp.

In yet another embodiment, the present invention provides a method for the use of these Designer Shrimp or other crustaceans, selectively enriched with certain health beneficial compounds, such as but not limited to, LC-PUFAs (DHA, ARA, EPA), carotenoids (e.g., lutein, β-carotene, astaxanthin, zeaxanthin), vitamins (e.g., vitamin A, vitamin C, vitamin E), minerals (e.g., iron, zinc, selenium, magnesium), or other beneficial compound as a food or feed for animals including man.

Production Methods

Completely contained shrimp culturing systems have been described (Leung and Moss 2000). Such systems that are known in the art can be used for the practice of this invention. Alternatively, semi-intensive production systems well known in the art can also be used (Haws and Boyd 2001). This invention provides the unexpected result that feeding shrimp certain types of feeds produces shrimp products selectively enriched in certain components of the feeds.

Flavor Enhancers

In the U.S., there is an emerging inland shrimp farming industry (Boyd 2001; Jory 2002) that uses fresh or low-salinity water to raise shrimp. Shrimp produced in such a fashion do not have the flavor profiles characteristic of wild-caught marine shrimp. This is primarily due to the absence of flavor agents, including iodine and bromophenols, contained in the natural marine food supply. Members of the bromophenol family can impart full fresh shrimp flavor (Boyle et al. 1992b) when present in the shrimp at the following levels: 2-bromophenol, 20 ppb; 3-bromophenol, 40 ppb; 2,6-dibromophenol, 4 ppb; and 2,4,6-tribromophenol, 20 ppb. Such levels in shrimp flesh can be achieved by as little as 10 ppm of Flavor Enhancer supplementation in the feed.

Desired flavor profiles can be obtained by the addition of one or more of these compounds in combination at various concentrations. These bromophenols can be produced synthetically or, alternatively, provided in the form of microbial or algal biomass. A number of algal strains have been demonstrated to produce significant amounts of these compounds. Such algal strains producing these bromophenols can be chosen from the following genera and/or species, but are not limited to these algae: Ulva, Ulva lactuca, Odonthalia, Odonthalia corymbifera, Symphocladia, Symphocladia laticscula, Codium, Codium fragile, Pterocladiella, Pterocladiella capillacea, Polysiphonia, and Polysiphonia sphaerocarpa. Using such a source for Flavor Enhancers could help in obtaining Organic certification for the shrimp produced, should this be desired.

This invention envisions feeding shrimp diets containing bromophenols at a level of between 10 mg to 1000 mg/ kg of feed. Preferably the level of enrichment will be such to add a bromophenol level in the animal of between 1 and 1000 ppb. The addition of these materials to the diet surprisingly improved the flavor of the shrimp.

Wild-caught shrimp and farmed shrimp from Asia may contain measurable levels of polychlorinated biphenols (PCB's), polyaromatic hydrocarbons (PAH's), and heavy metals (e.g., cadmium, mercury, lead), as well as residues of antibiotics banned for use in the U.S., the E.U., and Japan (e.g., chloramphenicol) (NRC 1999; Report 2001). For these reasons, in addition to rising consumer preference, farm raised shrimp are a rapidly growing part of U.S. and world agriculture. Much of the farm-raised shrimp available to consumers varies in taste, appearance, and nutritional characteristics. Improved, defined diets for shrimp can correct much of the inconsistency seen in the final product.

Organic Production Methods

A solution to improve consumer acceptance of aquacultured shrimp is the production of Organic Shrimp or 100% Organic Shrimp that are raised under controlled conditions and fed diets that consist entirely of certified Organic ingredients (within the limits set by the USDA). A major impediment to the production of Organic shrimp has been the availability of fishmeal and fish oil that is not derived from potentially contaminated or renewable marine sources. Until now, it has been believed that the addition of fishmeal and/or fish oil was a requirement for shrimp growth.

Prior to this invention, there have been no reports of a totally. vegetarian diet for shrimp (i.e., replacing the fishmeal and fish oil of the diet). The present invention describes for the first time a totally vegetarian diet for shrimp, wherein the fishmeal/fish oil is replaced by a combination of hydrolyzed plant protein and microalgae containing ω-3 LC-PUFAs. The inventors have surprisingly found that with the incorporation of certain components in the diet such as an algal source of; DHA, a diet that can support growth of marine animals in the absence of fishmeal and/or fish oil can be made. With the selection of dietary components that were themselves “Organic,” as defined by standard Organic certifying bodies such as the National Organic Program (NOP) or the like, these novel feeds could also be classified for the first time as “Organic Feeds.” The feeding of shrimp, using management practices known in the art that would also be considered “Organic” by the standard Organic certifying bodies such as NOP, would result in a shrimp that would have a unique composition, and be classified for the first time as an “Organic Shrimp” under the criteria of standardized Organic certification bodies such as NOP. Production of a 100% Organic Shrimp will require that all inputs be Organic certified with production and processing methods approved through the NOP. In embodiments, such feeds would include only non-genetically modified feed materials, no antibiotics, and no fishmeal or fish oil. In other embodiments, where the “Organic” label is not desired, genetically modified materials, antibiotics, and/or fishmeal and/or fish oil can be included. Such production has never before been achieved.

Pigment Enrichment

This invention also embodies the production of shrimp with a high level of-lutein and/or zeaxanthin. The main carotenoid of wild type or cultured shrimp is astaxanthin. This is a key pigment in the physiology and biochemistry of shrimp. There are no reports of shrimp where astaxanthin was not the predominant carotenoid of the animal. The inventors surprisingly found that modulation of the dietary carotenoids could result in a shrimp where the main carotenoid was not astaxanthin. Typically, astaxanthin comprises greater than 80% of the total carotenoid of the shrimp. The inventors have produced for the first time (see example 9) a shrimp with a significant modulation of the carotenoid profile where a carotenoid, naturally found at less than 5% of the total carotenoid fraction, was manipulated by diet to comprise greater than 30% of the improved shrimp. Dietary supplementation of the shrimp with certain materials including, but not limited to, marigold petals or extracts thereof, tomato products or extracts thereof, maize gluten or extracts thereof, certain microalgae such as, but not limited to, Chlorella, Spirulina, Dunaliella, Crypthecodinium, Schizochytrium, diatoms, or extracts thereof, can be used to elevate the levels of lutein, zeaxanthin, lycopene, and other carotenoids in the shrimp tissues. Surprisingly, these sources do not result in the complete conversion of the added carotenoids into astaxanthin.

The invention also envisions the use of artificial pigments including, but not limited to, lutein, zeaxanthin, lycopene, γ-carotene, and β-carotene, but the inclusion of these materials would not result in a 100% Organic certification of the feed or the shrimp. In both cases, however, a shrimp product will be produced which contains less than 80% the total carotenoids as astaxanthin, preferably less than 70%, and most preferably less than 60%, of the carotenoids as astaxanthin. Lutein, zeaxanthin or lycopene in their various forms are added to the standard feed to provide final carotenoid concentrations from 1 mg to 10 g per kg feed. Alternative carotenoids can be added to the diet, via various algal strains or synthetic methods, that are known to be present in crustaceans and are chosen from the following group, doradexanthin, idoxanthin, tetrol, α-cryptoxanthin, β-cryptoxanthin, echineone, 4-hydroxy-echineonone, canthaxanthin, β-apo-8′-carotenal, phoenicoxanthin, isocryptoxanthin, adonixanthin (Meyers and Latscha 1997). These can enhance the visual profile of the cultured crustaceans. Use of Organically certified algal and yeast strains that produce carotenoids, such as Haematococcus and Phaffia, can improve the nutritional value of Organically grown shrimp as well.

Fatty Acid Profile Enhancement

Shrimp are known to contain a small amount of DHA and typically have a DHA/EPA (eicosapentaenoic acid) ratio of 1:1 (USDA 2002a). However the total DHA content of a shrimp is typically less than 2.5 μg/g fresh weight of the shrimp (USDA 2002a). DHA has many specific health benefits and it would be beneficial to the consumer to elevate the DHA level of the shrimp. However, the use of fish oil or fishmeal to elevate DHA levels in the shrimp would result in a shrimp product that would no longer be certifiable as 100% Organic Shrimp by many Organic certifying bodies. Furthermore, the elevation of DHA via fish oil would be accompanied by an elevation of the EPA level. Elevated EPA levels are associated with reduced growth and increased bleeding times in humans and would therefore not be a beneficial attribute to the shrimp. The inventors surprisingly found that the feeding of a DHA source including, but not limited to, certain microalgae (e.g., Crypthecodinium, Schitzochytrium, Ulkenia, Parietochloris, etc.) or the extracts therefrom, particularly extracted phospholipids as described in U.S. Pat. No. 6,372,460, could effectively elevate the DHA levels of the shrimp to above 12.5 μg DHA/g fresh weight of shrimp and a DHA/EPA ratio greater than 1.0, more preferably to greater than 2.0, and most preferably to greater than 5.0. The source of DHA should be added at a level that provides DHA content in the feed from 5% of the total fat in the feed to 50% of the total fat in the feed. Since the microalgal biomass containing the DHA oil (as well as biomass from other algae containing ARA, EPA, and other LC-PUFAs) can be grown in defined conditions, the organisms are not GMO, materials can be certified Organic, and production methods can be certified Organic, compared to fish oil and fishmeal, this approach allows one to petition shrimp produced in the defined system for Organic certification.

Improved Cholesterol Content

The cholesterol content of shrimp and other shellfish is quite high. Levels of 1.5 to 2.0 mg total cholesterol/g fresh weight of shrimp are typically reported. Cholesterol is also considered an essential nutrient in the diet of shrimp, as there are reports that shrimp are unable to synthesize cholesterol de novo. As a result, it has heretofore been impossible to eliminate cholesterol from the diet of shrimp. The inventors have discovered that the use of certain phospholipids rich in DHA reduces and/or eliminates the need for cholesterol as a component of the diet. The use of these phospholipids alone or in conjunction with certain dietary fibers that include, but are not limited to, alginates, gums, starches, etc., can result in a reduction in the levels of cholesterol in the shrimp that have never before been achieved. Shrimp with levels of 0 to 1.25 mg cholesterol/g fresh weight are encompassed by this invention. Alternatively, the use of certain natural cholesterol-lowering agents, such as, but not limited to, red rice yeast (Monascus sp.), is also envisioned by this invention.

Finishing Feed

Finishing feeds or additives containing any of the enrichments described above can be provided throughout the culture of the shrimp. Alternatively and preferably, the finishing feeds or additives are provided from 1 to 60 days prior to harvest to provide the final enrichment and change in composition of the animals. Shrimp considered 100% Organic or Organic will need to be fed throughout the entire culture with the algal meal or extract replacement for the fishmeal or fish oil. Other enrichments can be limited to the final three weeks of the cultivation cycle. Certain embodiments of the invention will now be described in more detail through the following Examples. The Examples are intended solely to aid in more fully describing selected embodiments of the invention and should not be considered to limit the scope of the invention in any way.

### EXAMPLES

### Example 1

### Production of Shrimp Containing High Levels of DHA

Standard intensive and self-contained, semi-intensive, or extensive shrimp production systems can be used (Leung and Moss 2000; Haws and Boyd 2001). Intensive, zero-water exchange systems are preferable to produce Organic or 100% Organic high-DHA shrimp (Leung and Moss 2000). Two weeks prior to harvest date, the feeding regimen of the shrimp is altered to provide a Finishing Feed, which comprises the standard shrimp grow-out feed, plus a supplement of DHA (25 g DHA/kg feed, or 50% of total fat) provided as 200 g Crypthecodinium sp./kg feed (e.g., from AquaGrow Advantage; Advanced BioNutrition Corp., Columbia, Md.). Alternatively, 300 g Schizochytrium sp./kg of feed (5% of total fat of feed) can be used (e.g., from Aquafauna BioMarine, Hawthorne, Calif.). The high-DHA shrimp are harvested using processes and practices known in the art.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 2

### Production of a High-Lutein Shrimp

Standard intensive and self-contained, semi-intensive, or extensive shrimp production systems can be used (Leung and Moss 2000; Haws and Boyd 2001). Intensive, zero-water exchange systems (Leung and Moss 2000) would be preferable to produce an “Organic” high-lutein shrimp. Two weeks prior to harvest date, the feeding regimen of the shrimp is altered to provide a “Finishing Feed” which comprises the standard shrimp grow-out feed plus a supplement of lutein (60 mg lutein/kg feed) provided as a standardized marigold extract (6 mg lutein/100 mg oil) by the addition of 1 g marigold extract per kg feed. The high-lutein shrimp are harvested using processes and practices known in the art.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 3

### Production of an Aquacultured Shrimp with Improved Flavor

Standard intensive and self-contained, semi-intensive, or extensive shrimp production systems can be used (Leung and Moss 2000; Haws and Boyd 2001). Intensive, zero-water exchange systems (Leung and Moss 2000) are preferable to produce an Organic high-lutein shrimp. Two weeks prior to harvest date, the feeding regimen of the shrimp is altered to provide a Finishing Feed which comprises the standard shrimp grow-out feed plus a Flavor Enhancer, such as 2,6-dibromophenol (10 mg 2,6-dibromophenol/kg feed), by the addition of the flavor enhancer directly to the feed prior to extrusion or by coating the feed with the flavor enhancer using spray coating techniques standard in the art. The shrimp are harvested using processes and practices known in the art and will have a taste similar to marine-raised sea shrimp.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 4

### Production of a High-Lutein/High-DHA Shrimp

Standard intensive and self-contained, semi-intensive, or extensive shrimp production systems can be used (Leung and Moss 2000; Haws and Boyd 2001). Intensive, zero-water exchange systems as described in U.S. Pat. No. 6,327,996 would be preferable to produce an “Organic” high-lutein/high-DHA shrimp. Two weeks prior to harvest date, the feeding regimen of the shrimp is altered to provide a “Finishing Feed” which comprises the standard shrimp grow-out feed plus a supplement of lutein (60 mg lutein/kg feed) provided as a standardized marigold extract (6 mg lutein/100 mg oil) by the addition of 1 g marigold extract per kg feed and a supplement of DHA from Crypthecodinium added at a concentration of 50 g algal cells (at 20% DHA content) per kg feed. The high-lutein/high-DHA shrimp are harvested using processes and practices known in the art.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 5

### Production of a Low-Cholesterol Shrimp

Standard intensive and self-contained, semi-intensive, or extensive shrimp production systems can be used (Leung and Moss 2000; Haws and Boyd 2001). Intensive, zero-water exchange systems as described in U.S. Pat. No. 6,327,996 are preferable to produce an “Organic” high-lutein shrimp. Two weeks prior to harvest date, the feeding regimen of the shrimp is altered to provide a “Finishing Feed” which comprises the standard shrimp grow-out feed but with no added cholesterol and a source of DHA-containing phospholipids (e.g., AquaGrow DHA, a commercial product of Advanced Bionutrition Corp, Columbia, Md., USA) at a level of 50 g AquaGrow DHA/kg feed. The low cholesterol shrimp are harvested using processes and practices known in the art.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 6

### Production of Low Cholesterol Shrimp Using Red Rice Yeast (Monascus purpureus)

Standard intensive and self-contained, semi-intensive, or extensive shrimp production systems can be used (Leung and Moss 2000; Haws and Boyd 2001). Intensive, zero-water exchange systems (Leung and Moss 2000) would be preferable to produce an “Organic” high-lutein shrimp. Two weeks prior to harvest date, the feeding regimen of the shrimp is altered to provide a “Finishing Feed” which comprises the standard shrimp grow-out feed but with no added cholesterol and the addition of red rice yeast at a level of 50 g yeast per kg feed. The low cholesterol shrimp are harvested using processes and practices known in the art.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 7

### Production of 100% Organic Shrimp

An intensive, zero-water exchange production system (Leung and Moss 2000) is preferable to produce an “Organic” shrimp. Such as system would be managed under the guidelines of the NOP as a fully Organic operation. The feed input to the system is totally vegetarian. Fishmeal is replaced on a protein-to-protein basis with hydrolyzed non-GMO soy meal while fish oil is replaced on an oil for DHA basis with the microalgae Crypthecodinium. For a 1:1 replacement of the fish oil with algal oil, 115 g of Crypthecodinium biomass per kg of feed is used.

This certified Organic feed contains no antibiotics or other preservative chemicals. The shrimp are fed the Organic feed, which is produced in small particulate form for small shrimp and larger standardized pellets for larger shrimp using procedures standard in the industry. This Organic feed is used up to the time of harvest, unless an Organic Finishing Feed is utilized (as envisioned by this invention), The Organic shrimp are then harvested using processes and practices known in the art. These shrimp are distinguished biochemically by elevated DHA levels.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 8

### Production of Organic Shrimp

An intensive, zero-water exchange production system, such as that described by Leung and Moss (2000), is established and managed under the guidelines of the NOP as a fully Organic operation to produce “Organic” shrimp. The feed input to the system can be totally vegetarian. Fishmeal is replaced on a protein-to-protein basis with hydrolyzed non-GMO soy meal alone or in combination with other vegetable meals including but not limited to: whole wheat, corn gluten meal and pea meal while the fish oil is replaced with 115 g of Crypthecodinium biomass per kg of feed.

This certified Organic feed contains no antibiotics or other preservative chemicals. Ninety five percent of all components of the feed must be obtained from fully Organic certified sources. The shrimp are fed the Organic feed, which is produced in small particulate form for small shrimp and larger standardized pellets for larger shrimp using procedures standard in the industry. This Organic feed is used up to the time of harvest, unless an Organic Finishing Feed is utilized (as envisioned by this invention). The Organic shrimp are then harvested using processes and practices known in the art. By manipulation of the DHA-containing algal meal, organic shrimp could be made that could be distinguished biochemically by increased levels of DHA.

The shrimp so produced can be used as a healthy replacement for wild-caught or aquaculturally produced shrimp in any of the manners normally used to process shrimp including, but not limited to, steamed, cooked, cerveche, shredded, extruded, and dried.

### Example 9

### Shrimp with High Levels of Lutein

Juvenile shrimp (1-5 g) were cultured in 20 L tanks (5-10 shrimp per tank) at 23 degrees Celsius using standard shrimp diets (e.g., Rangen 35/2.5 shrimp diet). Test diets were prepared containing a 2:1 mixture of Rangen control feed with Lutein (Twin Labs) for a final lutein concentration in the diet of 8g/kg diet. Shrimp were fed at 0.3% body weight 2×/day for the duration of the experiment.

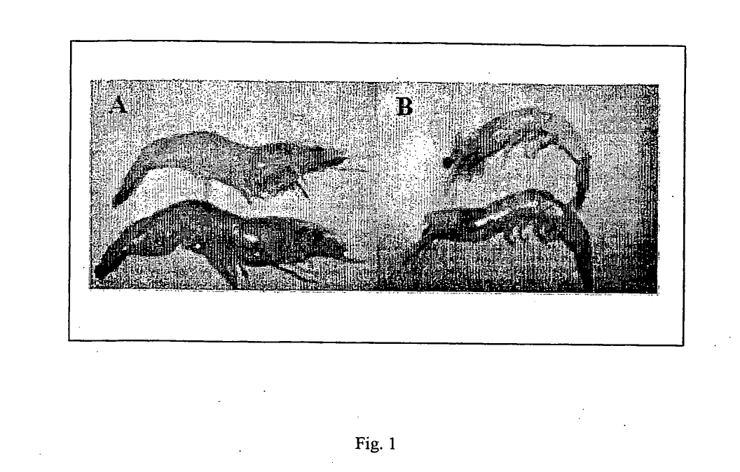

Following 14 days of feeding, the shrimp were collected, frozen to −20° C. and lyophilized prior to chemical analysis. A contract laboratory performed conventional HPLC carotenoid analysis with the results shown in Table 1 and FIG. 1. FIG. 1 demonstrates the effect of lutein enrichment on shrimp (A) before cooking and (B) after cooking. Non-enriched shrimp are shown in the top panels and their larger, more colorful, enriched counterparts are shown in the bottom panels.

TABLE 1
The carotenoid content of shrimp fed control diets compared to those fed
Designer Diets enriched with lutein.

| | AsX | Lutein | Other | Total | A/T | L/T | A/L | L + O/T |
|---|---|---|---|---|---|---|---|---|
| Control | 3.59 | 0.46 | 0.25 | 4.30 | 83% | 11% | 7.80 | 17% |
| | 4.63 | 0.10 | 0.35 | 5.08 | 91% | 2% | 46.30 | 9% |
| | 4.51 | 0.22 | 0.33 | 5.06 | 89% | 4% | 20.50 | 11% |
| | 4.09 | 0.33 | 0.27 | 4.69 | 87% | 7% | 12.39 | 13% |
| | 7.44 | 0.10 | 0.48 | 8.02 | 93% | 1% | 74.40 | 7% |
| | 5.84 | 0.06 | 0.59 | 6.49 | 90% | 1% | 97.33 | 10% |
| Mean | 5.02 | 0.21 | 0.38 | 5.61 | 89% | 4% | 43.12 | 11% |
| SD | 1.28 | 0.14 | 0.12 | 1.27 | 3% | 4% | 33.24 | 3% |
| Lutein | 6.45 | 1.70 | 1.38 | 9.53 | 68% | 18% | 3.79 | 32% |
| | 4.91 | 2.73 | 1.40 | 9.04 | 54% | 30% | 1.80 | 46% |
| | 4.63 | 9.39 | 1.47 | 15.49 | 30% | 61% | 0.49 | 70% |
| | 8.37 | 7.65 | 2.70 | 18.72 | 45% | 41% | 1.09 | 55% |
| | 6.82 | 1.71 | 1.15 | 9.68 | 70% | 18% | 3.99 | 30% |
| Mean | 6.24 | 4.64 | 1.62 | 12.49 | 53% | 33% | 2.23. | 47% |
| SD | 1.36 | 3.24 | 0.55 | 3.91 | 15% | 16% | 1.42 | 15% |

AsX = astaxanthin;
A (astaxanthin),
T (total carotenoids),
L (lutein).

### Example 10

### Shrimp with DHA Enrichment

Juvenile shrimp (1-5 g) were cultured in 20 L tanks (5-10 shrimp per tank) at 23° C. using standard shrimp diet. Shrimp were fed at 0.3% body weight 2×/day for the duration of the experiment. Control diets contained Ziegler SI-35 shrimp feed (Ziegler Bros., Gardners, Pa.) with 6.6 g DHA/kg feed (8.4% fat; 7.7% DHA in fat; 0.66% DHA in the diet). The test diet consisted of Ziegler SI-35 shrimp diet with AquaGrow® DHA (Advanced BioNutrition) replacing the fish oil component of the commercial diet.

Following 14 days of feeding with the finishing feed containing DHA, the shrimp were collected, frozen to −20° C. and lyophilized prior to chemical analysis. Fatty acid analysis was done according to routine procedures involving the preparation of fatty acid methyl esters followed by their separation and quantitation by gas chromatography. About 150 mg of shrimp tail muscle was accurately weighed into a 10 ml screw top tube and 1 mg of C15:0 (Supelco Co., Bellefonte, Pa.) was added as an internal standard. Two milliliters of Methanolic Base Reagent (Supelco Corp) was added, the sample was flushed with nitrogen, sealed and heated to 60° C. for 10 min. Samples were then cooled and 2.0 mL of water and 2.0 mL of hexane were added. Samples were vortex mixed then centrifuged. The fatty acid methyl esters in the hexane fraction were then separated by GC, using a Restek FAMEWAX (30 meter, 0.25 mm ID, 0.25 μm df) capillary column. The resulting percent DHA analyses are shown in Table 2.

[image]

### Example 11

### Shrimp with Low Cholesterol Levels

Juvenile shrimp (1-5 g) were cultured in 20 L tanks (5-10 shrimp per tank) at 23 degrees Celsius using standard shrimp diets (e.g., Rangen 35/2.5 shrimp diet). A newly prepared diet consisting of a 2:1 ratio of control diet to Red Rice Yeast (RRY; Monascus sp.) was prepared. Shrimp were fed both diets at 0.3% body weight 2×/day for the duration of the experiment.

Following 14 days of feeding this shrimp with the finishing feed containing DHA, the shrimp were collected, frozen to −20° C. and lyophilized prior to chemical analysis. Cholesterol analysis was done according to routine procedures involving extraction of the lyophilized shrimp muscle and a small amount of internal standard (cholestane) with chloroform/methanol (2:1). Samples were heated to 60° C. for 30 minutes to ensure complete extraction before cooling and diluting with an equal volume of water. The mixture was then centrifuged to establish a clear phase separation and the chloroform layer removed and dried under nitrogen. The dry precipitate was then dissolved in Ethanolic Base (2N NaOH in 95% ethanol) and heated to 60° C. for 30 minutes. Cool water was added to reduce the ethanol concentration to 66% (1:2 dilution with the ethanolic extract) and an equal volume of hexane added to extract the cholesterol. The hexane layer was then removed and dried under nitrogen. The dry sterol sample was then derivatized by adding 100 uL Tri-Sil Reagent (Supelco Corp). This material was evaporated under nitrogen and the sialilated sterols taken up in a small volume (100 uL) of hexane and applied directly to the gas chromatograph. A 50 m Supelco Wax column is typically used for the sterol separations as advised by the manufacturer. The resulting sterol analyses are shown in Table 3.

TABLE 3
Cholesterol Composition of Shrimp Fed Control Diets and Designer Diets
Enriched with Red Rice Yeast.

| | Control Shrimp | Shrimp with Red Rice Yeast |
|---|---|---|
| Cholesterol | 16.8% | 11.8% |
| (% of extractable lipid) | | |
| Cholesterol (mg/g) | 6.06 | 5.68 |

References

The specification is most thoroughly understood in light of the following references, all of which are hereby incorporated by reference in their entireties. The disclosures of the patents and other references cited above are also hereby incorporated by reference.

Boyd C (2001) Inland shrimp farming and the environment. World Aquaculture: 10-12.

Boyle J L, Lindsay R C, Stuiber D A (1992a) Bromophenol distribution in salmon and selected seafoods of fresh and saltwater origin. J Food Sci 57:918-922.

Boyle J L, Lindsay R C, Stuiber DA (1992b) Contributions of volatile bromophenols to marine associated flavors in fish and seafoods. J Aquatic Food Prod Tech 1:43-63.

Childs M T, Dorsett C S, King I B, Ostrander J G, Yamanaka W K (1990) Effects of shellfish consumption on lipoproteins in normolipidemic men. Am J Clin Nutr 51:1020-1027.

Connor W E, Lin D S (1982) The effect of shellfish in the diet upon the plasma lipid levels in humans. Metabolism 31:1046-1051.

De Oliveira e Silva E R, Seidman C E, Tian J J, Hudgins L C, Sacks F M, Breslow J L (1996) Effects of shrimp consumption on plasma lipoproteins. Am J Clin Nutr 64:712-717.

Haws M, Boyd C (2001) Methods for improving shrimp farming in Central America. Central American University Press, Managua.

Horrocks L A, Yeo Y K (1999) Health benefits of docosahexaenoic acid (DHA). Pharmacol Res 40:211-225.

Jory D (2002) Inland shrimp culture with zero water exchange ponds. Aquaculture Mag:74-77.

Kummer C (1992) Food: Farmed Fish. The Atlantic 270:88.

Leung P S, Moss S M (2000) Economic Assessment of a Prototype Biosecure Shrimp Growout Facility. In: Bullis R, Pruder G (eds) Controlled and Biosecure Production Systems: Evolution and Integration of Shrimp and Chicken Production Models. World Aquaculture Society, Sidney, Australia, pp 97-106.

Meyers S P, Latscha T (1997) Carotenoids. In: Abramo L, Conklin D, Akiyama D (eds) Crustacean Nutrition World Aquaculture Society, Baton Rouge, pp 164-194.

Middleditch B S et al. (1980) Metabolic profiles of penaied shrimp: dietary lipids and ovarian maturation. J Chromatogr 195:359-368.

NRC (1999) The Use of Drugs in Food Animals: Benefits and Risks. In. Committee on Drug Use in Food Animals, Panel on Animal Health, Food Safety, and Public Health, National Research Council, p 276.

Report G (2001) Federal oversight of seafood does not sufficiently protect consumers GAO-01-204. In. GAO, p 62.

Sylvia, Graham (1991) Consumer preferences, proximal analysis, and panel scores for three types of cultured and captured salmon. In: Pacific Fisheries Technologists Meeting, Victoria, B. C., Canada, p 26.

Tanaka K, Sakai T, Ikeda I, Imaizumi K, Sugano M (1998) Effects of dietary shrimp, squid and octopus on serum and liver lipid levels in mice. Biosci Bioteclinol Biochem 62:1369-1375.

Uauy-Dagach R, Valenzuela A (1992) Marine oils as a source of omega-3 fatty acids in the diet: how to optimize the health benefits. Prog Food Nutr Sci 16:199-243.

USDA (2002a) USDA Nutrient Database for standard reference, Release 15.

USDA (2002b) USDA website. http://www.usda.gov/.

We claim:

1. An aquaculturally-raised shrimp comprising DHA at a level higher than about 12.5 μg/g fresh weight.

2. The shrimp of claim 1, wherein the DHA level is higher than about 25 μg/g fresh weight.

3. The shrimp of claim 2, wherein the DHA level is higher than about 50 μg/g fresh weight.

4. An aquaculturally-raised shrimp comprising carotenoids, wherein astaxanthin comprises less than about 80% of the total carotenoids.

5. The shrimp of claim 4, wherein the non-astaxanthin carotenoids comprise one or more carotenoids chosen from β-carotene, γ-carotene, lutein, lycopene, zeaxanthin, and canthaxanthin.

6. An aquaculturally-raised shrimp comprising a lutein level higher than about 5 μ/g fresh weight.

7. An aquaculturally-raised shrimp comprising a Flavor Enhancer.

8. The shrimp of claim 7, wherein the Flavor Enhancer is chosen from bromophenol, 2,6-dibromophenol, 2,4,6-tribromophenol, and iodine.

9. The shrimp of claim 8, wherein the Flavor Enhancer comprises 2,6-dibromophenol at a level higher than about 0.06 μg per kilogram fresh weight.

10. The shrimp of claim 8, wherein the Flavor Enhancer comprises 2,4,6-tribromophenol at a level higher than about 6 μg per kilogram fresh weight.

11. The shrimp of claim 8, wherein the Flavor Enhancer comprises 2,6-dibromophenol and 2,4,6-tribromophenol at levels higher than about 0.06 and 6 μg per kilogram fresh weight.

12. An aquaculturally-raised shrimp comprising a cholesterol level lower than about 8.0 mg per gram fresh weight.

13. The shrimp of claim 12, wherein the cholesterol level is lower than about 6 mg per gram fresh weight.

14. The shrimp of claim 13, wherein the cholesterol level is lower than about 1.0 mg per gram fresh weight.

15. An aquaculturally-raised shrimp comprising a DHA/EPA ratio greater than about 2.0.

16. The shrimp of claim 15, comprising a DHA/EPA ratio greater than about 2.5.

17. The shrimp of claim 16, comprising a DHA/EPA ratio greater than about 5.0.

18. An aquaculturally-raised shrimp fed an exclusively vegetarian diet comprising hydrolyzed plant protein and microalgae.

19. An Organic shrimp.

20. A 100% Organic shrimp.

21. An aquaculturally-raised shrimp that has been certified as Organic by the United States Department of Agriculture.

22. An aquaculturally-raised shrimp that has been certified as 100% Organic by the United States Department of Agriculture.

23. A shrimp feed comprising red rice yeast.

24. The shrimp feed of claim 23, wherein comprising a Monascus sp. red rice yeast biomass chosen from a whole biomass, a lysed biomass, a fraction of a whole biomass, and a fraction of a lysed biomass.

25. The feed of claim 24, wherein the Monascus sp. comprises Monascus purpureus.

26. A shrimp feed comprising components chosen from DHA, lutein, lycopene, zeaxanthin, bromophenols, and chlorophyll.

27. The feed of claim 26, comprising from about 10 to about 1000 mg/kg 2,6,-dibromophenol.

28. The feed of claim 26, comprising from about 10 to about 1000 mg/kg 2,4,6-tribromophenol.

29. The feed of claim 26, comprising from about 10 to about 1000 mg/kg 2,6-dibromophenol, and from about 10 to about 1000 mg per kilogram 2,4,6-tribromophenol.

30. The feed of claim 26, comprising DHA, wherein the DHA level is greater than about 12.5 μg/g fresh weight.

31. The feed of claim 26, comprising lutein, wherein the lutein level is greater than about 5 μg/g fresh weight.

32. The feed of claim 26, comprising lycopene, wherein the lycopene level is greater than about 5 μg/g fresh weight.

33. The feed of claim 26, comprising zeaxanthin, wherein the zeaxanthin level is greater than about 6 μg/g fresh weight.

34. A method of producing an Organic shrimp comprising feeding microalgal DHA to shrimp.

35. A method of producing a shrimp comprising feeding to said shrimp one or more components chosen from microalgae enriched with DHA and microalgal extracts enriched with DHA.

36. The method of claim 35, wherein the microalgae are chosen from dinoflagelates and chitrids.

37. The method of claim 35, wherein the microalgae are chosen from Crypthecodinium sp., Crypthecodinium cohnii, Schizochytrium sp., Schizochytrium aggregatum, Schizochytrium aggregatum ATCC 28209, Thraustochytrium roseum ATCC 28210, Thraustochytrium sp. ATCC 26185, Thraustochytrium sp., Thraustochytrium visurgense ATCC 28208, Pavlova sp., Tetraselmis sp., and Isochrysis sp.

38. A method of producing a shrimp comprising providing the shrimp with a feed comprising a biomass enriched in one or more carotenoid.

39. The method of claim 38, wherein the biomass is chosen from one or more of microalgae, marigold extract, marigold petals, tomato extract, and processed tomato biomass.

40. A method of increasing the desirability of the flavor profile of a shrimp by adding one or more Flavor Enhancers to the shrimp's feed.

41. The method of claim 40, wherein the Flavor Enhancer comprises one or more bromophenols.

42. The method of claim 41, wherein the Flavor Enhancer is chosen from 2,6-dibromophenol and 2,4,6-tribromophenol.

43. A method of feeding a shrimp to a human or non-human animal, comprising providing for the animal's consumption a shrimp chosen from a high-DHA shrimp, a high carotenoid shrimp, a low cholesterol shrimp, and an Organic shrimp.
